(19) United States
(12) Patent Application Publication
(10) Pub. No.: US 2026/0118301 A1
(43) Pub. Date: Apr. 30, 2026

(54) SENSOR FOR MEASURING A GAS PROPERTY

(71) Applicant: Infineon Technologies AG, Neubiberg (DE)

(72) Inventors: Julia Isabel PEREZ BARRAZA, Unterhaching (DE); Malika BELLA, Ottobrunn (DE)

(21) Appl. No.: 19/320,698

(22) Filed: Sep. 5, 2025

(30) Foreign Application Priority Data
Sep. 18, 2024 (DE) 102024208941.9

(51) Int. Cl. G01N 25/18; G01N 33/00

(52) U.S. Cl. CPC G01N 25/18; G01N 33/0031; G01N 33/005

(57) ABSTRACT

A sensor for measuring a gas property includes a reference cavity and a measuring cavity, wherein a reference sensor element is arranged in the reference cavity, and a measuring sensor element is arranged in the measuring cavity. The measuring cavity is fluidly connected to ambient gas, the reference cavity is hermetically sealed, and a gettering material is arranged in the reference cavity and configured to absorb gas molecules inside the reference cavity.

### CROSS-REFERENCE TO RELATED APPLICATION

This application claims priority to Germany Patent Application No. 102024208941.9 filed on Sep. 18, 2024, the content of which is incorporated by reference herein in its entirety.

### TECHNICAL FIELD

The present disclosure relates to a sensor for measuring a gas property and a method for manufacturing a sensor for measuring a gas property.

### BACKGROUND

There is an increasing demand for reducing the consumption of petroleum and shifting to using green energy. For example, hydrogen generated by wind turbines is considered as a possible green fuel for automotive applications.

Sensors may be required to detect any leaking hydrogen to avoid the formation of Oxyhydrogen.

A highly sensitive hydrogen sensor to be operated at room temperature is disclosed in DE 10 2004 033597 A1. However, cars may be operated at temperatures well below and above room temperature.

A further sensor for measuring a gas property is disclosed in DE 10 2020 134 366 A1. The sensor for measuring a gas property, in particular a gas composition, more particularly a hydrogen level, comprises the semiconductor die, wherein the semiconductor die comprises a reference cavity and a measuring cavity. A reference sensor element is arranged in the reference cavity and a measuring sensor element is arranged in the measuring cavity. The reference cavity is sealed from ambient gas and the measuring cavity is fluidly connected to ambient gas. A fluid connection may relate to a connection allowing the passing of liquids and/or gas. For example, the reference cavity may be covered with a membrane allowing diffusion of gas into the measuring cavity.

Because of its light weight and small size, hydrogen exhibits one of the fastest diffusion rates in solid materials. In consequence, hydrogen intrusion into sealed cavities poses a problem for state-of-the-art hydrogen sensors with measuring and reference cavities as it can result in a lifetime drift eventually degrading the accuracy of the sensor.

### SUMMARY

There may be a need for a sensor for reliably measuring a gas property for automotive applications that overcomes the drawbacks of a reduced lifetime due to intrusion of unwanted gas molecules and atoms.

Subject-matter as defined in the independent claims is provided. Further implementations are described in the dependent claims.

Examples disclose a sensor for measuring a gas property, in particular a gas composition, more particularly a hydrogen level, wherein the sensor includes a reference cavity and a measuring cavity, wherein a reference sensor element is arranged in the reference cavity and a measuring sensor element is arranged in the measuring cavity. Therein, the measuring cavity is fluidly connected to ambient gas, while the reference cavity is sealed from the ambient gas, in particular it is hermetically sealed. Furthermore, a gettering material is arranged in the reference cavity and configured to absorb gas molecules inside the reference cavity.

Moreover, examples disclose a method for manufacturing one or more sensors for measuring a gas property, in particular a gas composition, in particular a hydrogen level, wherein the method includes: providing a semiconductor die including a reference cavity and a measuring cavity, arranging a reference sensor element inside the reference cavity, arranging a measuring sensor element inside the measuring cavity, depositing a gettering material inside the reference cavity, the gettering material being and configured to absorb gas molecules inside the reference cavity, fluidly connecting the measuring cavity to ambient gas, and sealing the reference cavity from the ambient gas, in particular hermitically sealing the reference cavity.

Those skilled in the art will recognize additional features and advantages upon reading the following detailed description, and upon viewing the accompanying drawings.

### BRIEF DESCRIPTION OF THE DRAWINGS

The present disclosure is illustrated by way of example, and not by way of limitation, in the figures of the accompanying drawings in which like reference numerals refer to similar or identical elements. The elements of the drawings are not necessarily to scale relative to each other. The features of the various illustrated examples can be combined unless they exclude each other.

FIG. 1 shows a wafer;

FIG. 2 shows the processed wafer of FIG. 1;

FIG. 3 shows the processed wafer of FIG. 2;

FIG. 4 shows the processed wafer of FIG. 3;

FIG. 5 shows the processed wafer of FIG. 4;

FIG. 6 shows a sensor for measuring a gas property;

FIG. 7 shows the sensor;

FIG. 8 illustrates a method for manufacturing a sensor.

### DETAILED DESCRIPTION

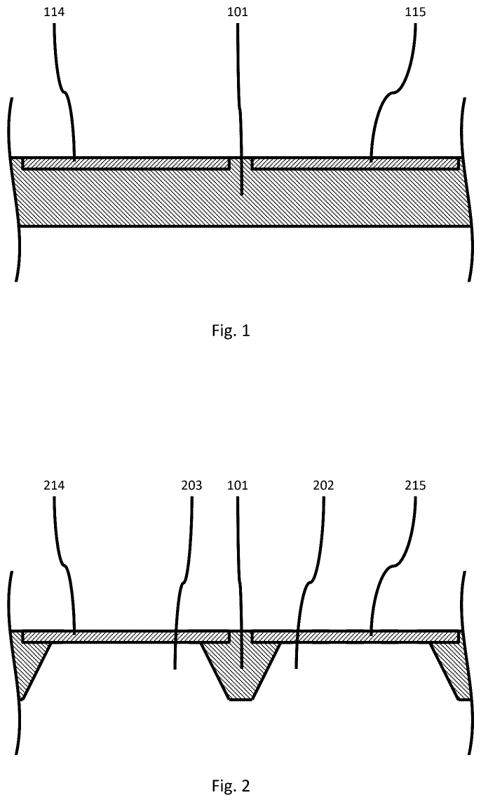

FIG. 1 shows a semiconductor wafer 101 with doped wells 114 and 115. The doped wells 114 and 115 have a doping type opposite to the doping type of the semiconductor wafer 101. The wells 114 and 115 are provided at the front side of the semiconductor wafer 101.

As shown in FIG. 2, at least one reference cavity 202 and at least one measuring cavity 203 are etched in the backside of the semiconductor wafer 101 to form membranes 214 and 215. The large back side cavities and may be formed using a pn-etch. The wells 114 and 115 may be n-doped and the semiconductor wafer may be a p-doped silicon wafer.

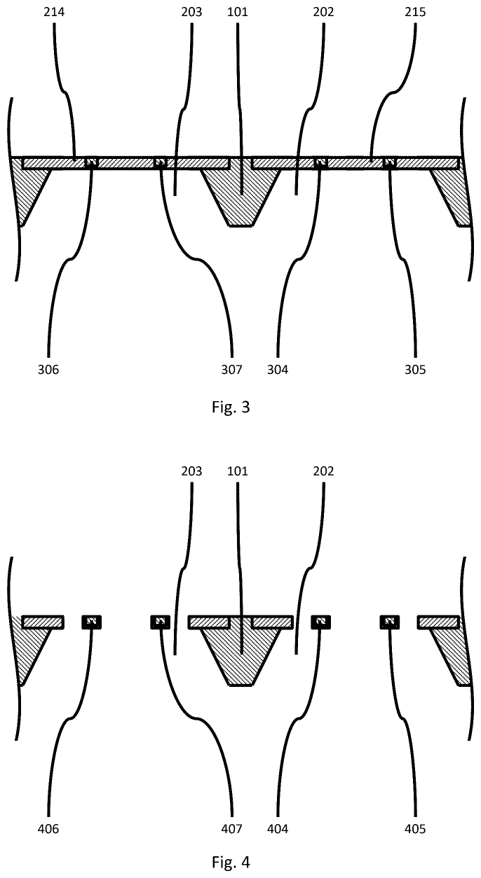

FIG. 3 shows the wafer 101 after conductive regions 304, 305, 306, 307 have been formed within the surface of the membranes 214 and 215. The conductive regions 304, 305, 306, 307 may be formed by doping. Alternatively or in addition, they may be formed by depositing a conductive material.

As shown in FIG. 4, two reference sensor elements 404, 405 as well as two measuring sensor elements 406, 407 may be formed from the conductive regions 304, 305, 306, 307. For example, the reference sensor elements 404, 405 and the measuring sensor elements 406, 407 may be wire elements. The wires may be formed from the membranes 214 and 215 by using the Bosch etch. The process may also be referred to as a release of the wires.

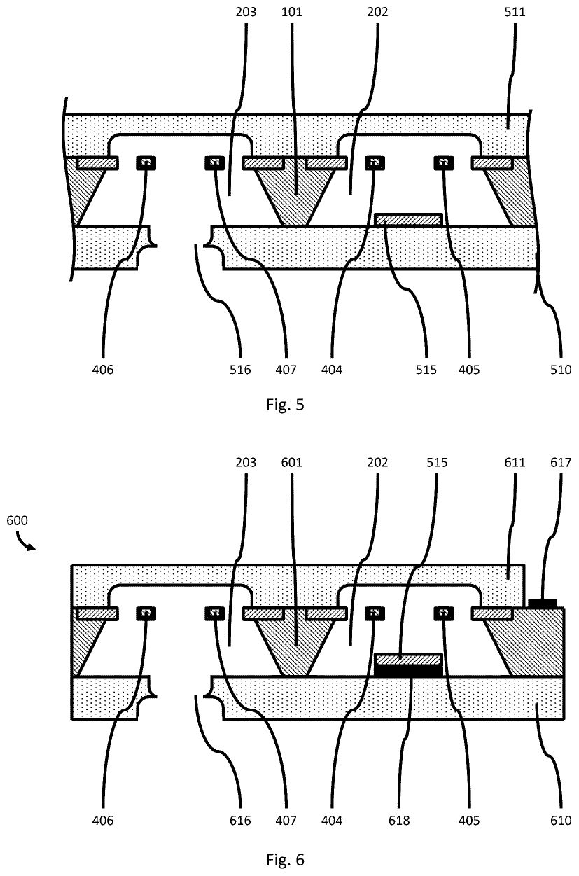

FIG. 5 shows that two covering wafers 510 and 511 are bonded to the semiconductor wafer 101 for sealing, in particular hermetically sealing, the reference cavity 202 and covering the measuring cavity 203. The covering wafers 510, 511 can be silicon or glass wafers. An opening 516 is provided as in inlet port within one of the two covering wafers 510, 511 that allows ambient gas to enter the measuring cavity 203. In other words, the opening 516 enables that ambient gas is fluidly connected to the measuring cavity 203. The sealed reference cavity 202 can be filled with a reference gas such as nitrogen, for instance.

Furthermore, a gettering material 515 may be formed on or within a surface of one of the two covering wafers 510, 511 such that the gettering material 515 is arranged within the reference cavity 202. For example, the gettering material 515 is deposited on a top surface of the bottom covering wafer 510 facing the reference cavity 202. Alternatively, the gettering material can be placed on a surface or within the semiconductor wafer 101, e.g., next to a reference sensor element 404, 405. The gettering material 515 may be configured to absorb gas molecules inside the reference cavity 202. In particular, the gettering material can be configured to absorb hydrogen and/or water molecules inside the reference cavity at an operational temperature. Moreover, the gettering material can be configured not to absorb nitrogen at the operational temperature. The operational temperature of the sensor 600 can correspond or be close to an ambient temperature of the sensor 600. In this case, the gettering material 515 is configured to getter hydrogen and/or water at or around the ambient temperature. Alternatively, as some materials getter hydrogen at different temperatures above the ambient temperature, the sensor 600 can further comprise a heater structure that is in thermal contact with the gettering material 515 and operable to elevate a temperature of the gettering material 515 to a gettering temperature. For example, the gettering material 515 can be or comprise titanium, which getters hydrogen in a temperature range from about 20° C. to 400° C. while it getters other gas molecules such as nitrogen, oxygen and carbon dioxide only at temperatures above 700° C., hence not gettering the reference gas at an operational temperature of the sensor 600, which is typically around an ambient temperature. The gettering material 515 can further be or comprise at least one of: zirconium, palladium, a metallic alloy, and an organic compound, such as 1,4-diphenyl butadiyne (DPB) and its derivatives. The gettering material 515 can be configured to maintain a purity level of the reference gas of at least 99.5%, in particular at least 99.9%. The gettering material 515 can be deposited via lithography or via inkjet printing, for instance.

Further, the semiconductor wafer 101 with the bonded covering wafers 510, 511 may be diced to form one or more of the sensors 600 shown in FIG. 6. The one or more sensors 600 may thus be formed using established semiconductor manufacturing processes rendering the manufacture of the described sensors very cost effective.

FIG. 6 shows the sensor 600 for measuring a gas property. The sensor 600 is in particular configured for measuring a gas composition, for example, a hydrogen level. The sensor 600 comprises a semiconductor die 601. A reference cavity 202 and a measuring cavity 203 are provided in the semiconductor die 601. As explained herein before, the reference cavity 202 and the measuring cavity 203 may have been formed by etching. A reference sensor element 404 is arranged in the reference cavity 202 and a corresponding measuring sensor element 406 is arranged in the measuring cavity 203. As shown in FIG. 6, two reference sensor elements 404, 405 may be provided in the reference cavity 202 and correspondingly, two measuring sensor elements 406, 407 may be provided in a measuring cavity 203.

The reference cavity 202 is sealed from ambient gas. In particular, the reference cavity 202 may be hermetically sealed from ambient gas. The reference cavity 202 can be filled with a reference gas, e.g., nitrogen or air. On the other hand, the measuring cavity 203 is fluidly connected to ambient gas. In particular, an opening 616 configured as an inlet may be provided for the purpose.

The reference sensor elements 404, 405 and/or the measuring sensor elements 406, 407 may be formed as one piece with the semiconductor die 601. This may facilitate manufacturing of the sensor 600. Moreover, it may lead to the reference sensor elements 404, 405 having the same property as the measuring sensor elements 406, 407.

Sensors for measuring a gas property, which may also be called gas sensors, may have a cross-sensitivity to different environment characteristics, such as humidity, temperature, flow, and concentration of the gas to be sensed. Typically, dedicated sensors for these additional properties may have to be included in order to differentiate the signal of interest. For example, the complementary temperature sensor may have to be added. This may lead to a complex device, where different dice or sensing elements have to be combined inside the package.

The sensors as disclosed herein may be fabricated with two identical sensing elements (e.g., the reference sensor element and the measuring sensor element) in one die. One element (e.g., the measuring sensor element) is exposed to the ambient of interest and the other element (e.g., the reference sensor element) is enclosed within a hermetically sealed cavity (e.g., the reference cavity). Hence, the package complexity may be reduced. Further, the device sensitivity may be improved.

For example, a differential read out between the two sensor elements (e.g., the reference sensor element and the measuring sensor element) may significantly reduce or even eliminate cross-sensitivity to temperature, as well as other sources of error and operational drift.

Moreover, the arrangement of a gettering material 515 within the reference cavity 202 can ensure a high purity of the reference gas within the reference cavity 202 by gettering water molecules and/or hydrogen molecules that due to their high diffusivity may still be able to enter the sealed reference cavity 202. This in turn ensures a high stability of the reference signal picked up by the reference sensor element 404, 405, which can be critical for an accurate reading of the sensor 600. The gettering material 515 can further reduce a lifetime drift of the sensor due to a changing reference signal from hydrogen or other molecules entering the reference cavity 202 and hence impurifying the reference gas in the reference cavity 202 over its lifetime.

The sensor 600 may comprise a covering 610, 611 for sealing the reference cavity 202 and covering the measuring cavity 203. The opening 616 may be provided in the covering 710 to provide a fluid connection of the measuring cavity 203 to ambient gas. In implementations, the covering 710 and/or the covering 711 may be formed from silicon or glass. The opening 616 may be formed by etching the glass covering. Techniques for etching glass are well established in semiconductor manufacturing processes. The reference cavity 202 may be filled with a gas, in particular inert gas, more particularly with at least one of Nitrogen and Xenon. During manufacturing of the sensor 600, in particular, during bonding of the coverings 610, 611 to the semiconductor die 601, a specific gas pressure may be applied, which will be present in the reference cavity 202 afterwards. In particular, the gas pressure in the reference cavity may be below 10 mbar. This may be considered as vacuum.

The coverings 610, 611 may be hermetically bonded to the semiconductor die 601. Thus, no gas may enter the reference cavity 202 or the measuring cavity 203 via the interface of the semiconductor die 601 with the coverings 610, 611. Several techniques for hermetically bonding the coverings 610, 611 to the semiconductor die 601 may be used. For example, glass frit may be used for bonding. Other techniques include metal bonding or soldering. In examples, an adhesive-free bonding technique may be used. Anodic bonding has proven to be a suitable technique for bonding the coverings 610, 611 to the semiconductor die 601.

The reference sensor element 404 and the measuring sensor element 406 may have the same structure. In particular, the reference sensor element 404 may be formed from the same material as the measuring sensor element 406 and may have the same geometry. Thus, the only difference between the reference sensor element 404 and the measuring sensor element 406 may be that one is provided in the reference cavity 202 and the other one in the measuring cavity 203. In particular, the reference sensor element 404, the measuring sensor element 406 and the semiconductor die 601 may be formed as one piece. Likewise, the reference sensor element 405 may correspond to the measuring sensor element 407.

The semiconductor die 601 may comprise an integrated circuit 617. The reference sensor element 404, 405 and/or the measuring sensor element 406, 407 may be part of the integrated circuit 617. Thus, means for reading out the reference sensor element 404, 405 and the measuring sensor element 406, 407 may be directly integrated with the semiconductor die 601. In particular, amplifiers may be provided close to the reference sensor element 404, 405 and the measuring sensor element 406, 407 to avoid noise in the sending signals. The integrated circuit 617 may further be connected to an optional heater structure 618 that is in thermal contact with the gettering material 515.

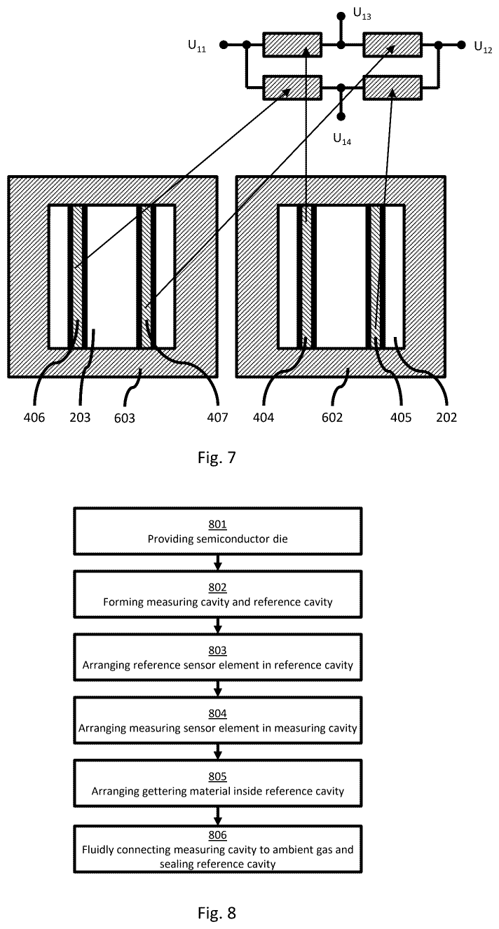

As shown in FIG. 7, the sensor 600 may comprise at least two reference sensor elements 404, 405 and at least two measuring sensor elements 406, 407 forming a full bridge, e.g., in a Wheatstone bridge configuration. In this example, the reference cavity 202 is comprised by a first semiconductor die 602 and the measuring cavity 203 is comprised by a second semiconductor die 603 different from the first semiconductor die 602. In particular, a first one 404 of the two reference sensor elements 404, 405 and a first one 406 of the two measuring sensor elements 406, 407 form a first half bridge between a first node U11 and a second node U22. Accordingly, a second one 405 of the two reference sensor elements 404, 405 and a second one 407 of the two measuring sensor elements 406, 407 form a second half bridge between the first node U11 and the second node U22 in parallel to the first half bridge as illustrated in FIG. 7. A supply voltage can be applied during a measurement phase between the first node U11 and the second node U12, while the bridge voltage is measured across a third node U13 and a fourth node U14, each arranged in between the reference sensor element 404, 405 and the measuring sensor element 406, 407 of each respective half bridge.

Due to the provision of the reference sensor elements in the reference cavity, the sensor 600 may be suitable to be operated between −40° C. and 150° C.

In examples, the reference sensor element and the measuring sensor element may be formed as corresponding membranes. Alternatively, as shown in the figures, the reference sensor elements and the measuring sensor elements may be formed as corresponding wires. These may be linear wires or meander wires forming resistive elements.

For example, the reference sensor elements 404, 405 and the measuring sensor elements 406, 407 may be configured for measuring a gas concentration via thermal conductivity. For example, the reference sensor elements 404, 405 and the measuring sensor elements 406, 407 may comprise silicon wires etched on a thin membrane. The silicon wires may be doped to increase the electrical conductance.

The proposed sensor may be particularly useful for automotive powertrains based on hydrogen fuel cells. For example, a sensor of the described type may be located near an exhaust of the fuel cell, in order to control the fuel cell. Heretofore, the sensor may be configured to determine an H2 content of 0 to 40%.

Furthermore, the sensor may be located next to the high pressure H2 tank. The sensor may be configured for sensing an H2 leakage. For this purpose, the sensor may have a sensitivity for a concentration of 0 to 4% H2. In other examples, the sensor may be located close to a battery pack. The sensor may be configured for detecting out gassing of H2 due to the battery pack being overloaded and/or damaged. For this purpose, the sensor may detect H2 with a concentration of 0 to 4%.

FIG. 8 illustrates a method of manufacturing a sensor. The method comprises a first step 801 of providing a semiconductor die and a second step 802 of forming reference and measuring cavities 202, 203. The first and second steps 801, 802 can comprise providing a semiconductor wafer, providing wells on a surface of the wafer, performing back side etching for defining the reference cavity 202 and the measuring cavity 203. The method further comprises a third step 803 of arranging a reference sensor element 404, 405 in the reference cavity 202, and a fourth step 804 of cavity 203. The method further comprises a third step 803 of arranging a measuring sensor element 406, 407 in the measuring cavity 203. The third and fourth steps 803, 804 can comprise a release etch of a membrane to form resistive sensor elements 404, 405, 406, 407, for instance. The method further comprises a step 805 of arranging a gettering material 515 inside the reference cavity 202, and a step 806 of fluidly connecting the measuring cavity 203 to ambient gas and sealing the reference cavity 202.

Although specific examples have been illustrated and described herein, it will be appreciated by those of ordinary skill in the art that a variety of alternate and/or equivalent implementations may be substituted for the specific examples shown and described without departing from the scope of the present implementation. This application is intended to cover any adaptations or variations of the specific examples discussed herein. Therefore, it is intended that this implementation be limited only by the claims and the equivalents thereof.

It should be noted that the methods and devices including its preferred implementations as outlined in the present document may be used stand-alone or in combination with the other methods and devices disclosed in this document. In addition, the features outlined in the context of a device are also applicable to a corresponding method, and vice versa. Furthermore, all aspects of the methods and devices outlined in the present document may be arbitrarily combined. In particular, the features of the claims may be combined with one another in an arbitrary manner.

It should be noted that the description and drawings merely illustrate the principles of the proposed methods and systems. Those skilled in the art will be able to implement various arrangements that, although not explicitly described or shown herein, embody the principles of the implementation and are included within its spirit and scope. Furthermore, all examples and implementations outlined in the present document are principally intended expressly to be only for explanatory purposes to help the reader in understanding the principles of the proposed methods and systems. Furthermore, all statements herein providing principles, aspects, and implementations of the implementation, as well as specific examples thereof, are intended to encompass equivalents thereof.

In particular, the following examples are disclosed:

### Example 1

A sensor (600) for measuring a gas property comprising a reference cavity (202) and a measuring cavity (203), wherein a reference sensor element (404, 405) is arranged in the reference cavity (202), a measuring sensor element (406, 407) is arranged in the measuring cavity (203), the measuring cavity (203) is fluidly connected to ambient gas, the reference cavity (202) is hermetically sealed, and wherein a gettering material (515) is arranged in the reference cavity (202) and configured to absorb gas molecules inside the reference cavity (202).

### Example 2

The sensor (600) according to the preceding example, wherein the sensor (600) is configured to measure a gas composition, in particular a hydrogen level of the ambient gas.

### Example 3

The sensor (600) according to one of the preceding examples, wherein the gettering material (515) is configured to absorb hydrogen and/or water molecules inside the reference cavity (202) at an operational temperature.

### Example 4

The sensor (600) according to one of the preceding examples, wherein the gettering material (515) is configured not to absorb nitrogen at an operational temperature.

### Example 5

The sensor (600) according to example 3 or 4, wherein the operational temperature is an ambient temperature.

### Example 6

The sensor (600) according to one of the preceding examples, wherein the gettering material (515) comprises titanium (Ti), zirconium (Zr), palladium (Pd), a metallic alloy and/or an organic compound.

### Example 7

The sensor (600) according to one of the preceding examples, wherein the reference cavity (202) is filled with a reference gas, in particular with nitrogen.

### Example 8

The sensor (600) according to example 7, wherein the gettering material (515) is configured to maintain a purity level of the reference gas of at least 99.5%, in particular at least 99.9%.

### Example 9

The sensor (600) according to one of the preceding examples, wherein the measuring sensor element (406, 407) and the reference sensor element (404, 405) have the same structure.

### Example 10

The sensor (600) according to one of the preceding examples, wherein the semiconductor die (601) comprises an integrated circuit (617) and wherein the reference sensor element (404, 405) and/or the measuring sensor element (406, 407) are part of the integrated circuit (617).

### Example 11

The sensor (600) of any one of the preceding examples, wherein the sensor (600) comprises at least two reference sensor elements (404, 405) arranged in the reference cavity (202) and at least two measuring sensor elements (406, 407) arranged in the measuring cavity (203), the at least two reference sensor elements (404, 405) and the at least two measuring sensor elements (406, 407) are electrically connected in a Wheatstone bridge configuration, a first one of the two reference sensor elements (404, 405) and a first one of the two measuring sensor elements (406, 407) form a first half bridge of the Wheatstone bridge configuration, and a second one of the two reference sensor elements (404, 405) and a second one of the two measuring sensor elements (406, 407) form a second half bridge of the Wheatstone bridge configuration.

### Example 12

The sensor (600) of any one of the preceding examples, wherein the reference sensor element (404, 405) and the measuring sensor element (406, 407) are formed as corresponding membranes (214, 215) or wires.

### Example 13

The sensor (600) of any one of the preceding examples, wherein the reference sensor element (404, 405) and the measuring sensor element (406, 407) are formed as resistive elements.

### Example 14

The sensor (600) of any one of the preceding examples, wherein the sensor (600) is a thermal conductivity sensor.

### Example 15

The sensor (600) of any one of the preceding examples, further comprising a semiconductor die (601) including the reference cavity (202) and the measuring cavity (203).

### Example 16

The sensor (600) of any of examples 1 to 14, further comprising a first semiconductor die (602) including the reference cavity (202) and a second semiconductor die (603) including the measuring cavity (203).

### Example 17

The sensor (600) of any of the preceding examples, further comprising a heater structure (618) arranged in the reference cavity (202) in thermal contact with the gettering material (515).

### Example 18

A method of manufacturing a sensor (600) for measuring a gas concentration, the method comprising:
providing a semiconductor die (601) comprising a reference cavity (202) and a measuring cavity (203),
arranging a reference sensor element (404, 405) inside the reference cavity (202),
arranging a measuring sensor element (406, 407) inside the measuring cavity (203),
depositing a gettering material (515) inside the reference cavity (202), the gettering material (515) being and configured to absorb gas molecules inside the reference cavity (202), fluidly connecting the measuring cavity (203) to ambient gas, and
hermetically sealing the reference cavity (202).

### Example 19

The method according to example 18, wherein depositing the gettering material (515) comprises printing, in particular inkjet printing.

### Example 20

The method according to example 18, wherein depositing the gettering material (515) comprises performing a lithography process.

### Example 21

The method according to any of examples 18 to 20, wherein depositing the gettering material (515) is performed on a glass or silicon surface of the reference cavity (202).

1. A sensor (600) for measuring a gas property comprising a reference cavity (202) and a measuring cavity (203), wherein
a reference sensor element (404, 405) is arranged in the reference cavity (202),
a measuring sensor element (406, 407) is arranged in the measuring cavity (203),
the measuring cavity (203) is fluidly connected to ambient gas,
the reference cavity (202) is hermetically sealed, and
wherein a gettering material (515) is arranged in the reference cavity (202) and configured to absorb gas molecules inside the reference cavity (202).

2. The sensor (600) according to the preceding claim, wherein the sensor (600) is configured to measure a gas composition, in particular a hydrogen level of the ambient gas.

3. The sensor (600) according to one of the preceding claims, wherein the gettering material (515) is configured to absorb hydrogen and/or water molecules inside the reference cavity (202) at an operational temperature.

4. The sensor (600) according to one of the preceding claims, wherein the gettering material (515) is configured not to absorb nitrogen at an operational temperature.

5. The sensor (600) according to claim 3 or 4, wherein the operational temperature is an ambient temperature.

6. The sensor (600) according to one of the preceding claims, wherein the gettering material (515) comprises Ti, Zr, Pd, a metallic alloy and/or an organic compound.

7. The sensor (600) according to one of the preceding claims, wherein the reference cavity (202) is filled with a reference gas, in particular with nitrogen.

8. The sensor (600) according to claim 7, wherein the gettering material (515) is configured to maintain a purity level of the reference gas of at least 99.5%, in particular at least 99.9%.

9. The sensor (600) according to one of the preceding claims, wherein the measuring sensor element (406, 407) and the reference sensor element (404, 405) have the same structure.

10. The sensor (600) according to one of the preceding claims, wherein the semiconductor die (601) comprises an integrated circuit (617) and wherein the reference sensor element (404, 405) and/or the measuring sensor element (406, 407) are part of the integrated circuit (617).

11. The sensor (600) of any one of the preceding claims, wherein
the sensor (600) comprises at least two reference sensor elements (404, 405) arranged in the reference cavity (202) and at least two measuring sensor elements (406, 407) arranged in the measuring cavity (203),
the at least two reference sensor elements (404, 405) and the at least two measuring sensor elements (406, 407) are electrically connected in a Wheatstone bridge configuration,
a first one of the two reference sensor elements (404, 405) and a first one of the two measuring sensor elements (406, 407) form a first half bridge of the Wheatstone bridge configuration, and
a second one of the two reference sensor elements (404, 405) and a second one of the two measuring sensor elements (406, 407) form a second half bridge of the Wheatstone bridge configuration.

12. The sensor (600) of any one of the preceding claims, wherein the reference sensor element (404, 405) and the measuring sensor element (406, 407) are formed as corresponding membranes (214, 215) or wires.

13. The sensor (600) of any one of the preceding claims, wherein the reference sensor element (404, 405) and the measuring sensor element (406, 407) are formed as resistive elements.

14. The sensor (600) of any one of the preceding claims, wherein the sensor (600) is a thermal conductivity sensor.

15. The sensor (600) of any one of the preceding claims, further comprising a semiconductor die (601) including the reference cavity (202) and the measuring cavity (203).

16. The sensor (600) of any of claims 1 to 14, further comprising a first semiconductor die (602) including the reference cavity (202) and a second semiconductor die (603) including the measuring cavity (203).

17. The sensor (600) of any of the preceding claims, further comprising a heater structure (618) arranged in the reference cavity (202) in thermal contact with the gettering material (515).

18. A method of manufacturing a sensor (600) for measuring a gas concentration, the method comprising:
providing a semiconductor die (601) comprising a reference cavity (202) and a measuring cavity (203),
arranging a reference sensor element (404, 405) inside the reference cavity (202),
arranging a measuring sensor element (406, 407) inside the measuring cavity (203),
depositing a gettering material (515) inside the reference cavity (202), the gettering material (515) being and configured to absorb gas molecules inside the reference cavity (202),
fluidly connecting the measuring cavity (203) to ambient gas, and
hermetically sealing the reference cavity (202).

19. The method according to claim 18, wherein depositing the gettering material (515) comprises printing, in particular inkjet printing.

20. The method according to claim 18, wherein depositing the gettering material (515) comprises performing a lithography process.

21. The method according to any of claims 18 to 20, wherein depositing the gettering material (515) is performed on a glass or silicon surface of the reference cavity (202).
